(19) United States
(12) Patent Application Publication
(10) Pub. No.: US 2018/0071209 A1
(43) Pub. Date: Mar. 15, 2018

(54) AQUEOUS SOLUTION OF POLYMERS

(71) Applicants: Dow Global Technologies LLC, Midland, MI (US); Regents of the University of Michigan, Ann Arbor, MI (US)

(72) Inventors: Elizabeth Tocce, Midland, MI (US); Susan L. Jordan, Collegeville, PA (US); Desai Kashappa Goud, Rockville, MD (US); Steven Schwendeman, Ann Arbor, MI (US)

(21) Appl. No.: 15/558,811

(22) PCT Filed: Mar. 23, 2016
(86) PCT No.: PCT/US16/23663

§ 371 Date: Sep. 15, 2017

Related U.S. Application Data
(60) Provisional application No. 62/137,450, filed on Mar. 24, 2015.

(51) Int. Cl. A61K 9/00; A61K 9/08; A61K 47/38; A61K 47/34

(52) U.S. Cl. CPC A61K 9/0043; A61K 47/34; A61K 47/38; A61K 9/08

(57) ABSTRACT

Provided is an aqueous composition comprising
(a) 0.5% to 5% one or more dissolved cellulose derivative, by weight based on the weight of the solution, and
(b) 1% to 10% one or more dissolved polyvinyl caprolactam-polyvinyl acetate-polyethylene glycol graft copolymer, by weight based on the weight of the solution;
   wherein said aqueous solution has complex viscosity of 20 mPa*s or less at 25° C.

Compositions for application to nasal mucosae, such as pharmaceutical compositions for transmucosal delivery of physiologically active agents, are often desirable. Nasal sprays are drug delivery systems intended for administration to the nasal cavity. However, known nasal sprays often rapidly exit the nasal cavity either via dripping from the nostrils or via the back of the nasal cavity into the nasopharynx, which can lead to insufficient efficacy of the physiologically active agent(s). High-viscosity delivery systems, such as ointments or gels, are retained in the nasal cavity for a longer time period, but the exact dosage of ointments and gels is difficult to meter and subsequently deliver to the desired location within the nasal cavity.

US 2013/0157963 describes a topical ophthalmic composition. It would be desirable to provide a composition that had relatively low viscosity prior to contact with nasal mucosal tissue in order to be suitable for spraying and that had relatively high viscosity after coming into contact with nasal mucosal tissue.

The following is a statement of the invention.

An aspect of the present invention is an aqueous composition comprising
(a) 0.5% to 10% one or more dissolved cellulose derivative, by weight based on the weight of the solution, and
(b) 1% to 10% one or more dissolved polyvinyl caprolactam-polyvinyl acetate-polyethylene glycol graft copolymer, by weight based on the weight of the solution; wherein said aqueous solution has complex viscosity of 25 mPa*s or less at 25° C.

The following is a brief description of the FIGURE.

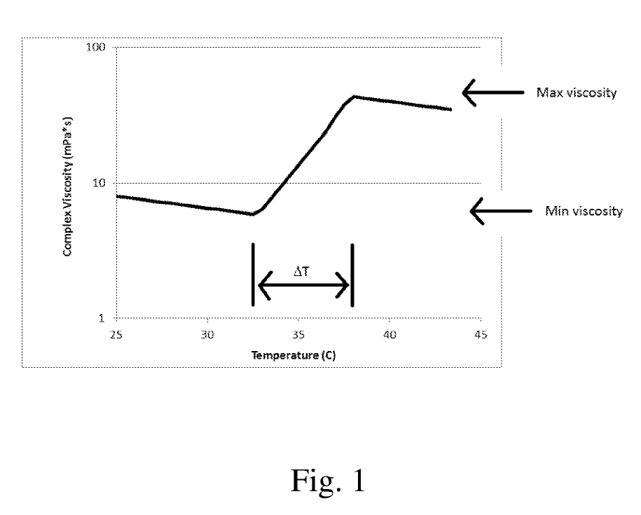

FIG. 1 shows a schematic curve of complex viscosity versus temperature, showing how to identify the maximum viscosity, the minimum viscosity, and ΔT.

The following is a detailed description of the invention.

As used herein, the following terms have the designated definitions, unless the context clearly indicates otherwise.

A compound is considered herein to be cationic if an atom or a chemical group that bears a positive charge is covalently bound to the compound. A cationic functional group is an atom or a chemical group that bears a positive charge.

Cellulose is a naturally occurring organic polymer consisting of linear chain of linked D-glucose units. Cellulose is often reacted with one or more of various reagents to produce a derivative in which one or more of the hydroxyl atoms on the cellulose is replaced with one or more functional groups. One class of useful cellulose derivatives is the class of water-soluble cellulose derivatives, which are compounds that are soluble in water at 25° C. in the amount of 1 gram or more per 100 grams of water. An amount of polymer is considered herein to be dissolved in water if the mixture of that amount of the polymer and water forms a stable composition that is not hazy to the unaided eye and that does not show phase separation of the polymer from the water.

As used herein, an aqueous composition is a composition that contains 50% or more water by weight based on the weight of the aqueous solution.

As used herein, complex viscosity is measured by oscillation of a cone and plate fixture at 0.5 Pa of oscillating stress at 0.5 cycles per second. Any cone angle may be chosen as long as the measurement is made under conditions in which complex viscosity does not change if oscillating stress is varied from 0.3 Pa to 0.8 Pa.

As defined herein, “gelation temperature” of a composition is determined as follows, as illustrated in FIG. 1. The complex viscosity is observed as a function of temperature. For purposes of the present invention, the temperature range of interest is 20° C. to 45° C. Compositions that have a gelation temperature show, over the temperature range of interest, the following behavior: as temperature increases, complex viscosity decreases relatively slowly, then complex viscosity increases relatively quickly, then complex viscosity again decreases relatively slowly. Outside of the temperature range of interest, the complex viscosity versus temperature may or may not show other behaviors. In the temperature range of interest, the point of minimum viscosity is identified, and the temperature at that point (TMIN) is noted, along with the value of the viscosity at that point (VMIN). Also in the temperature range of interest, the point of maximum viscosity is identified, and the temperature at that point (TMAX) is noted, along with the value of the viscosity at that point (VMAX). The parameter ΔT=TMAX−TMIN. The viscosity rise quotient is VRISE=VMAX/VMIN. The composition is said herein to have a gelation temperature if VRISE is 3 or larger and ΔT is 15° C. or smaller. The gelation temperature is defined as TGEL=0.5*(TMAX+TMIN).

Methylcellulose (MC) polymer compound that has repeat units of the structure I:

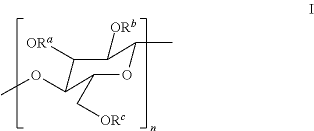

In structure I, the repeat unit is shown within the brackets. The index n is sufficiently large that structure I is a polymer; that is, n is sufficiently large that the “2% solution viscosity” (as defined below) of the compound is 2 mPa*s or higher. In MC, —R^{a}, —R^{b}, and —R^{c} is each independently chosen from —H and —CH₃. The choice of —R^{a}, —R^{b}, and —R^{c} may be the same in each repeat unit, or different repeat units may have different choices of —R^{a}, —R^{b}, and —R^{c}.

Methylcellulose polymer is characterized by the weight percent of methoxyl groups. The weight percentages are based on the total weight of the methylcellulose polymer. By convention, the weight percent is an average weight percentage based on the total weight of the cellulose repeat unit, including all substituents. The content of the methoxyl group is reported based on the mass of the methoxyl group (i.e., —OCH₃). The determination of the % methoxyl in methylcellulose (MC) polymer is carried out according to the United States Pharmacopeia (USP 37, “Methylcellulose”, pages 3776-3778).

Methylcellulose polymer is also characterized by the viscosity of a 2 wt.-% solution in water at 20° C. The 2% by weight methylcellulose polymer solution in water is prepared and tested according to United States Pharmacopeia (USP 37, “Methylcellulose”, pages 3776-3778). As described in the United States Pharmacopeia, viscosities of less than 600 mPa·s are determined by Ubbelohde viscosity measurement and viscosities of 600 mPa·s or more are determined using a Brookfield viscometer. When the 2 wt-% solution of MC has been made, the correct viscometer chosen, and the viscosity measured, the resulting measured viscosity is known herein as the “2% solution viscosity.”

Hydroxypropyl methylcellulose polymer has the structure I, where —R^{a}, —R^{b}, and —R^{c} is each independently chosen from —H, —CH₃, and structure II:

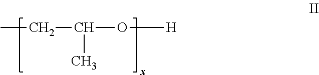

The choice of —R^{a}, —R^{b}, and —R^{c} may be the same in each repeat unit, or different repeat units may have different choices of —R^{a}, —R^{b}, and —R^{c}. The number x is an integer of value 1 or larger. One or more of —R^{a}, —R^{b}, and —R^{c} has structure II on one or more of the repeat units.

Hydroxypropyl methylcellulose polymer is characterized by the weight percent of methoxyl groups. The weight percentages are based on the total weight of the hydroxypropyl methylcellulose polymer. By convention, the weight percent is an average weight percentage based on the total weight of the cellulose repeat unit, including all substituents. The content of the methoxyl group is reported based on the mass of the methoxyl group (i.e., —OCH₃). The determination of the % methoxyl in hydroxypropyl methylcellulose polymer is carried out according to the United States Pharmacopeia (USP 37, “Hypromellose”, pages 3296-3298).

Hydroxypropyl methylcellulose polymer is characterized by the weight percent of hydroxypropyl groups. The weight percentages are based on the total weight of the hydroxypropyl methylcellulose polymer. The content of the hydroxypropoxyl group is reported based on the mass of the hydroxypropoxyl group (i.e., —O—C₃H₆OH). The determination of the % hydroxypropoxyl in hydroxypropyl methylcellulose (HPMC) is carried out according to the United States Pharmacopeia (USP 37, “Hypromellose”, pages 3296-3298).

Hydroxypropylmethylcellulose polymer is also characterized by the viscosity of a 2 wt. % solution in water at 20° C. The 2% by weight hydroxypropylmethylcellulose polymer solution in water is prepared and tested according to United States Pharmacopeia (USP 37, “Hypromellose”, pages 3296-3298). As described in the United States Pharmacopeia, viscosities of less than 600 mPa·s are determined by Ubbelohde viscosity measurement and viscosities of 600 mPa·s or more are determined using a Brookfield viscometer. This viscosity is known herein as the “2% solution viscosity.”

Sodium carboxymethyl cellulose (sodium CMC) has structure I in which —R^{a}, —R^{b}, and —R^{c} is each independently chosen from —H and —CH₂COONa. The choice of —R^{a}, —R^{b}, and —R^{c} may be the same in each repeat unit, or different repeat units may have different choices of —R^{a}, —R^{b}, and —R^{c}. The average number of groups per D-glucose unit in which —R^{a}, —R^{b}, or —R^{c} is —H (denoted “x”) is 1.5 to 2.8. The average number of groups per D-glucose unit in which —R^{a}, —R^{b}, or —R^{c} is —CH₂COONa (denoted “y” or “degree of substitution”) is 0.2 to 1.5. In sodium CMC, x+y is 3.0. Sodium CMC is characterized by the viscosity (Brookfield LVT at 25° C.) of a 2% solution by weight in water.

Cationic HEC has structure I in which —R^{a}, —R^{b}, and —R^{c} is each independently chosen from —H and —(CH₂CH₂O)ₙQ, where n is 1 to 5 and Q is a cationic functional group. Preferably, the cationic functional group Q has the structure V

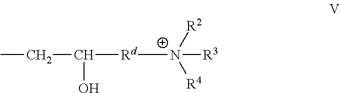

where —R^{d}— is a bivalent organic group. Cationic HEC is characterized by the viscosity (Brookfield LVT at 25° C.) of a 2% solution by weight in water. Cationic HEC is also characterized by the % Nitrogen as measured by the Kjeldahl nitrogen test.

As used herein, a PC-PA-PEG graft copolymer is a polyvinyl caprolactam-polyvinyl acetate-polyethylene glycol graft copolymer. PC-PA-PEG graft copolymer has one or more polyethylene glycol moiety covalently bonded to a polymer that contains polymerized units of vinyl acetate and polymerized units of vinyl caprolactam.

The composition of the present invention contains one or more cellulose derivative. Preferred cellulose derivatives are soluble in water. Preferred cellulose derivatives are methylcellulose (MC), hydroxypropyl methylcellulose (HPMC), sodium carboxymethyl cellulose (NaCMC), cationic hydroxyethyl cellulose (CHEC), and mixtures thereof. More preferred cellulose derivatives are HPMC and MC.

Among MC polymers, preferably the % methoxyl is 15% or higher; more preferably 25% or higher. Among MC polymers, preferably the % methoxyl is 40% or lower; more preferably 35% or lower. Among MC polymers, preferably the viscosity of a 2 weight % solution in water is preferably 2 mPa*s or higher; more preferably 4 mPa*s or higher. Among MC polymers, preferably the viscosity of a 2 weight % solution in water is preferably 10,000 mPa*s or lower; more preferably 6,000 mPa*s or lower.

Among HPMC polymers, preferably the % methoxyl is 10% or higher; more preferably 18% or higher. Among HPMC polymers, preferably the % methoxyl is 30% or lower; more preferably 26% or lower. Among HPMC polymers, preferably the % hydroxypropyl is 4% or higher; more preferably 6% or higher. Among HPMC polymers, preferably the % hydroxypropyl is 20% or lower; more preferably 15% or lower.

Among HPMC polymers, preferably the viscosity of a 2 weight % solution in water is preferably 2 mPa*s or higher; more preferably 4 mPa*s or higher. Among HPMC polymers, preferably the viscosity of a 2 weight % solution in water is preferably 20,000 mPa*s or lower; more preferably 5,000 mPa*s or lower. Preferably, when HPMC polymer is used that has viscosity of a 2 weight % solution in water of 2,000 mPa*s or higher, the amount of HPMC polymer, by weight based on the weight of the composition, is 2% or lower.

Among sodium CMC polymers, preferably the degree of substitution is 0.95 or lower. Preferably the degree of substitution of sodium CMC is 0.75 or higher, more preferably 0.8 or higher. Preferably, the viscosity of sodium CMC solution (2% by weight in water at 25° C.) is 200 mPa*s or higher; more preferably 400 mPa*s or higher. Preferably, the viscosity of sodium CMC solution (2% by weight in water at 25° C.) is 1500 mPa*s or lower; more preferably 1000 mPa*s or lower.

Among cationic HEC polymers, preferably, —R^{d}— is a hydrocarbon group with 1 to 8 carbon atoms; more preferably with 1 to 2 carbon atoms; more preferably with 1 carbon atom. Preferably, —R², —R³, and —R⁴ is each independently a substituted or unsubstituted hydrocarbon group. Preferably —R², —R³, and —R⁴ are all unsubstituted hydrocarbon groups; more preferably R², R³, and R⁴ are all unsubstituted alkyl groups; more preferably R², R³, and R⁴ are all methyl groups. Preferred cationic HEC has viscosity of a 2% solution by weight in water of 50 mPa*s or higher; more preferably 100 mPa*s or higher; more preferably 200 mPa*s or higher. Preferred cationic HEC has viscosity of a 2% solution by weight in water of 2,000 mPa*s or lower; more preferably 900 mPa*s or lower. Preferred cationic HEC has % nitrogen of 1.2 or higher; more preferably 1.4 or higher. Preferred cationic HEC has % nitrogen of 3 or lower; more preferably 2.5 or lower.

The composition of the present invention contains one or more PC-PA-PEG graft copolymer, which is a polyvinyl caprolactam-polyvinyl acetate-polyethylene glycol graft copolymer. Preferred PC-PA-PEG graft copolymers are soluble in water. Preferably, the PC-PA-PEG graft copolymer has a polyethylene glycol (PEG) backbone with one or two side chains. Preferably, the PEG backbone has average molecular weight of 1,000 or more; more preferably 3,000 or more. Preferably, the PEG backbone has average molecular weight of 20,000 or less; more preferably 10,000 or less. Preferably, each side chain is a random copolymer of vinyl acetate and N-vinyl caprolactam.

Preferably, the PC-PA-PEG graft copolymer has average molecular weight of 30,000 or higher; more preferably 50,000 or higher; more preferably 70,000 or higher. Preferably, the PC-PA-PEG graft copolymer has average molecular weight of 1,000,000 or lower; more preferably 500,000 or lower; more preferably 200,000 or lower.

Preferably, the amount of PEG backbone in the PC-PA-PEG graft copolymer is, by weight based on the weight of the PC-PA-PEG graft copolymer, 3% or more; more preferably 5% or more; more preferably 7% or more. Preferably, the amount of PEG backbone in the PC-PA-PEG graft copolymer is, by weight based on the weight of the PC-PA-PEG graft copolymer, 50% or less; more preferably 35% or less; more preferably 25% or less.

Preferably, the amount of polymerized units of vinyl acetate in the PC-PA-PEG graft copolymer is, by weight based on the weight of the PC-PA-PEG graft copolymer, 5% or more; more preferably 10% or more; more preferably 15% or more. Preferably, the amount of polymerized units of vinyl acetate in the PC-PA-PEG graft copolymer is, by weight based on the weight of the PC-PA-PEG graft copolymer, 70% or less; more preferably 60% or less; more preferably 50% or less.

Preferably, the amount of polymerized units of vinyl caprolactam in the PC-PA-PEG graft copolymer is, by weight based on the weight of the PC-PA-PEG graft copolymer, 10% or more; more preferably 20% or more; more preferably 30% or more. Preferably, the amount of polymerized units of vinyl caprolactam in the PC-PA-PEG graft copolymer is, by weight based on the weight of the PC-PA-PEG graft copolymer, 90% or less; more preferably 80% or less.

The amount of cellulose derivative in the composition of the present invention is preferably, by weight based on the weight of the composition, 0.02% or more; more preferably 0.05% or more; more preferably 0.09% or more. The amount of cellulose derivative in the composition of the present invention is preferably, by weight based on the weight of the composition, 10% or less; more preferably 7% or less; more preferably 4% or less; more preferably 2% or less.

The amount of PC-PA-PEG graft copolymer in the composition of the present invention is preferably, by weight based on the weight of the composition, 1% or more; more preferably 2% or more; more preferably 4% or more. The amount of PC-PA-PEG graft copolymer in the composition of the present invention is preferably, by weight based on the weight of the composition, 15% or less; more preferably 10% or less; more preferably 7% or less.

Preferably the composition of the present invention exhibits a gelation temperature of 39° C. or less, more preferably 37° C. or less. The gelation temperature of the composition of the present invention is preferably at least 24° C., more preferably at least 26° C., more preferably at least 28° C., and most preferably at least 30° C.

Preferably, the composition of the present invention exhibits VRISE of 2 or higher; more preferably 3 or higher; more preferably 4 or higher; more preferably 5 or higher. Preferably, the composition of the present invention exhibits VRISE of 10 or lower. Preferably, the composition of the present invention exhibits ΔT of 5° C. or more. Preferably, the composition of the present invention exhibits ΔT of 20° C. or less; more preferably 15° C. or less. Preferably, the composition of the present invention exhibits TGEL of 37° C. or below; more preferably 36° C. or below. Preferably, the composition of the present invention exhibits TGEL of 33° C. or above.

The composition of the present invention has complex viscosity at 25° C. of 20 mPa*s or lower; preferably 15 mPa*s or lower; more preferably 10 mPa*s or lower. Preferably, the composition of the present invention has complex viscosity at 37° C. of 25 mPa*s or higher; more preferably 30 mPa*s or higher; more preferably 40 mPa*s or higher; more preferably 50 mPa*s or higher.

For making a solution of a cellulose derivative in water, a preferred method is to bring the cellulose derivative into contact with liquid water to make a mixture and then provide mechanical agitation to the mixture. Preferably, the mixture has temperature of 80° C. or higher; more preferably 90° C. or higher. After the cellulose derivative is dissolved, the mixture is preferably cooled to 25° C.

The composition of the present invention is very useful for application to nasal mucosa, e.g. for transmucosal delivery of a physiologically active agent. A low viscosity at 5° C. or 20° C., i.e., at a temperature at which the composition is usually stored and/or applied, facilitates the release of the composition from a container comprising such composition, e.g. by spraying, and the administration of the composition to nasal mucosa. The temperature of the composition increases after its application to nasal mucosa. It is contemplated that this temperature increase will cause the temperature to rise above the gelation temperature, causing the composition to rise in viscosity. It is expected that the rise in viscosity will facilitate retention of the composition of the present invention on the nasal mucosa.

Preferably the composition of the present invention contains one or more physiologically active agents, preferably one or more physiologically active agents selected from the following: one or more drugs; one or more diagnostic agents; or one or more essential oils; or one or more physiologically active agents that are useful for cosmetic or nutritional purposes. The term “drug” denotes a compound having beneficial prophylactic and/or therapeutic properties when administered to an individual, typically a mammal, especially a human individual. Physiologically active agents that are useful for intranasal delivery are known in the art.

Some physiologically active agents and some methods of intranasal delivery are described in WO 2015/009799.

The composition of the present invention is particularly useful for intranasal delivery of one or more physiologically active agents or for delivery through a mucosal membrane located in the nasal cavity, such as drugs utilized in therapies for allergic rhinitis, nasal congestion and infections, in treatments of diabetes, migraine, nausea, smoking cessation, acute pain relief, nocturnal enuresis, osteoporosis, vitamin B-12 deficiency, and for administering intranasal vaccine such as, for example, influenza vaccine; however, the physiologically active agents are not limited to these examples. Especially preferred drugs are acetaminophen, azelastine hydrochloride, beclomethasone dipropionate monohydrate, sumatriptan succinate, dihydroergotamine mesylate, fluticasone propionate, triamcinolone acetonide, budesonide, fentanyl citrate, butorphanol tartrate, zolmitriptan, desmopressin acetate hydrate, salmon calcitonin, nafarelin acetate, buserelin acetate, elcatonin, oxytocin, insulin, mometasone furoate, estradiol, metoclopramide, xylometazoline hydrochloride, ipratropium bromide hydrate, olopatadine hydrochloride, oxymetazoline hydrochloride, dexpanthenol, hydrocortisone, naphazoline hydrochloride, phenylephrine hydrochloride, mepyramine maleate, phenylephrine hydrochloride, cromolyn sodium, levocabastine hydrochloride, vitamin B12, prednisolone sodium metasulphobenzoate, naphazoline nitrate, tetrahydrozoline hydrochloride, chlorpheniramine maleate, benzethonium chloride, ketotifen fumarate, histamine dihydrochloride, fusafungine, or combinations thereof. Examples of essential oils are menthol, methyl salicylate, thymol, eucalyptus oil, camphor, anise, sweet orange, or combinations thereof.

Preferred embodiments of the present invention will possess a variety of benefits. The fact that the viscosity is higher at 37° C. than at 25° C. will mean that the composition may be easily applied at 25° C., for example to the interior or the nasal cavity, by various methods, for example by spraying, and then when the composition comes into contact with living tissue at 37° C., the viscosity will rise, which will enable the composition to stay in the nasal cavity without running out due to gravity. The longer residence time in contact with the mucosal membrane will allow physiologically active agents more opportunity to penetrate into the tissue and/or the bloodstream. Additionally, it is expected that preferred embodiments of the present invention have one or more of the following benefits. Preferably, the composition of the present invention will act to improve the solubilization of physiologically active agents; will moderate the swelling rate of the composition; will adhere well to mucosal membrane; and/or will delay mucociliary clearance.

When the composition of the present invention does not contain a physiologically active agent, the composition of the present invention is useful, for example, for rinsing and/or moisturizing the nasal cavity.

The composition for transmucosal delivery further comprises a liquid diluent, of which at least 55 weight percent and up to 100 percent is water. The composition of the present invention may additionally comprise an organic liquid diluent; however, the composition of the present invention should comprise at least 55, preferably at least 65, more preferably at least 75, more preferably at least 90, and more preferably at least 95 weight percent of water, based on the total weight of the organic liquid diluent and water. The composition of the present invention preferably contains up to 45, more preferably up to 35, more preferably up to 25, more preferably only up to 10, and more preferably only up to 5 weight percent of an organic liquid diluent, based on the total weight of the organic liquid diluent and water. In one embodiment the diluent consists of water. The water is typically a high-quality grade of water such as purified water, for example USP purified water, PhEur purified water or water for Injection (WFI).

The term “organic liquid diluent” as used herein means an organic solvent or a mixture of two or more organic solvents that is liquid at 25° C. and atmospheric pressure. Preferred organic liquid diluents are polar organic solvents having one or more heteroatoms, such as oxygen, nitrogen or halogen (like chlorine). More preferred organic liquid diluents are alcohols, for example multifunctional alcohols, such as propylene glycol, polyethylene glycol, polypropylene glycol and glycerol; or preferably monofunctional alcohols, such as ethanol, isopropanol or n-propanol; or acetates, such as ethyl acetate. More preferably the organic liquid diluents have 1 to 6, most preferably 1 to 4 carbon atoms. The organic liquid diluent is preferably pharmaceutically acceptable, such as ethanol or glycerol.

The composition of the present invention may comprise one or more optional adjuvants, such as one or more suspending agents, odor, flavor or taste improvers, preservatives, pharmaceutically acceptable surfactants, coloring agents, opacifiers, or antioxidants. Typically, pharmaceutically acceptable optional adjuvants are selected.

For stability purposes, compositions of the invention (for example intranasal compositions) may be protected from microbial or fungal contamination and growth by inclusion of one or more preservatives. Examples of pharmaceutically acceptable anti-microbial agents or preservatives may include, but are not limited to, quaternary ammonium compounds (e.g. benzalkonium chloride, benzethonium chloride, cetrimide, cetylpyridinium chloride, lauralkonium chloride and myristyl picolinium chloride), mercurial agents (e.g. phenylmercuric nitrate, phenylmercuric acetate and thimerosal), alcoholic agents (e.g. chlorobutanol, phenylethyl alcohol and benzyl alcohol), antibacterial esters (e.g. esters of para-hydroxybenzoic acid), chelating agents such as disodium edetate (EDTA) and other anti-microbial agents such as chlorhexidine, chlorocresol, sorbic acid and its salts (such as potassium sorbate) and polymyxin. Examples of pharmaceutically acceptable anti-fungal agents or preservatives may include, but are not limited to, sodium benzoate, sorbic acid, sodium propionate, methylparaben, ethylparaben, propylparaben and butylparaben. The preservative(s), if included, are typically present in an amount of from 0.001 to 1%, such as from 0.015% to 0.5%, based on the total weight of the composition. Preferably, the preservative is selected from benzalkonium chloride, EDTA and/or potassium sorbate. More preferably, the preservative is EDTA and/or potassium sorbate.

The following are examples of the present invention.

The following materials were used:
MC1=METHOCEL™ A4M methylcellulose polymer, from Dow Chemical Company, % methoxyl substitution 27.5 to 31.5%, viscosity of 2% by weight solution in water, 2,663 to 4,970 mPa*s.
HPMC1=METHOCEL™ K4M hydroxypropyl methylcellulose polymer, from Dow Chemical Company, % methoxyl substitution 19.0-24.0%, % hydroxypropoxyl substitution 7.0-12.0%, viscosity of 2% by weight solution in water, 2,663 to 4,970 mPa*s.
NaCMC1=WALOCEL™ CRT 1000 PA sodium carboxymethylcellulose polymer, from Dow Chemical Company, 0.9 degree of substitution, viscosity of 2% by weight solution in water of 550-800 mPa*s.
CHEC1=UCARE™ JR 400 cationic HEC polymer from Dow Chemical Company, 1.2-2.2% Nitrogen, viscosity of 2% by weight aqueous solution in water of 300-500 mPa*s.
Graft1=SOLUPLUS™ copolymer from BASF, a polyvinyl caprolactm-polyvinyl acetate-polyethylene glycol graft copolymer (13% PEG 6000, 57% vinyl caprolactam, 30% vinyl acetate), having PEG 6000 backbone with one or two side chains consisting of random copolymer of vinyl acetate and vinyl caprolactam, with average molecular weight of 118,000.

Solutions were made by the following procedure: Stock polymer solutions of methylcellulose (MC, Methocel™ A4M cellulose ether polymer) and hydroxypropyl methylcellulose (HPMC, Methocel™ K4M cellulose ether polymer) were prepared by a hot and cold technique. The required quantities of polymers (1.5 w/w) were added to double-distilled water (ddH₂O) previously maintained at 80° C. under constant stirring with an overhead mixer at 1000 rpm. The polymer-water mixtures were continuously stirred for 10 min at 80° C., then at 4-8° C. for 20 min. Sodium carboxymethylcellulose (sodium CMC, Walocel™ CRT 1000 PA cellulose ether polymer) solution was prepared by adding the required quantity of polymer (3% w/w) to ddH₂O, and then stirred on a hot plate with a stir bar maintaining a small vortex indention for 6-8 h at room temperature. Cationic hydroxyethyl cellulose (cationic HEC, UCARE™ JR 400 cellulose ether polymer) was prepared in a two-step procedure. First, the polymer was dissolved in ddH₂O (3% w/w) at room temperature until the solution appeared transparent (approximately 1 h) and then gradually heated to 65° C. and maintained for 1 h stirring at 1000 rpm. All polymer solutions were then hydrated at 4° C. for 24 h to facilitate sufficient hydration of polymers and remove air bubbles. Polymer solution concentrations were prepared by diluting with ddH₂O. Stock solutions of Soluplus™ PC-PA-PEG graft copolymer, were prepared by adding a desired quantity of polymer to ddH₂O at room temperature and allowing to continuously stir on a hot plate with a small indentation/vortex for 48 h. Solutions containing more than one polymer component were prepared by adding desired quantities of stock polymer solutions to a vial and adding sufficient ddH₂O to achieve desired polymer concentrations.

Each solution was measured for complex viscosity as a function of temperature from 22° C. to 40° C. at 1° C./minute. The gelation temperature TGEL was assessed for each solution.

The results were as follows (Examples denoted “-C” are comparative examples).

| | | | | | | TGEL |
|---|---|---|---|---|---|---|
| Example | MC1⁽¹⁾ | HPMC1⁽¹⁾ | NaCMC⁽¹⁾ | CHEC1⁽¹⁾ | GRAFT1⁽¹⁾ | (° C.) |
| 1C | 0.1 | 0 | 0 | 0 | 0 | none⁽²⁾ |
| 2C | 0 | 0.1 | 0 | 0 | 0 | none⁽³⁾ |
| 3C | 0 | 0 | 0.1 | 0 | 0 | none⁽³⁾ |
| 4C | 0 | 0 | 0 | 0.1 | 0 | none⁽³⁾ |
| 5 | 0.1 | 0 | 0 | 0 | 5 | 34 |
| 6 | 0 | 0.1 | 0 | 0 | 5 | 35 |
| 7 | 0 | 0 | 0.1 | 0 | 5 | 35 |
| 8 | 0 | 0 | 0 | 0 | 5 | 38 |
| 9C | 0 | 0 | 0 | 0 | 0 | none⁽²⁾ |

⁽¹⁾abbreviations are defined above; amounts are in weight %
⁽²⁾has a TGEL greater than 50° C.
⁽³⁾no TGEL observed

Further results were as follows:

| | | | | | | TGEL |
|---|---|---|---|---|---|---|
| Example | MC1⁽¹⁾ | HPMC1⁽¹⁾ | NaCMC⁽¹⁾ | CHEC1⁽¹⁾ | GRAFT1⁽¹⁾ | (° C.) |
| 10C | 0.25 | 0 | 0 | 0 | 0 | none⁽³⁾ |
| 11 | 0.25 | 0 | 0 | 0 | 5 | 32 |
| 12C | 0 | 0.25 | 0 | 0 | 0 | none⁽³⁾ |
| 13 | 0 | 0.25 | 0 | 0 | 5 | 31.5 |
| 14C | 0 | 0 | 0.25 | 0 | 0 | none⁽³⁾ |
| 15 | 0 | 0 | 0.25 | 0 | 5 | 36.5 |
| 16C | 0 | 0 | 0 | 0.25 | 0 | none⁽³⁾ |
| 17 | 0 | 0 | 0 | 0.25 | 5 | 37 |

⁽¹⁾amount in weight %
⁽²⁾has a TGEL greater than 50° C.
⁽³⁾no TGEL observed

1. An aqueous composition comprising
(a) 0.5% to 5% one or more dissolved cellulose derivative, by weight based on the weight of the solution, and
(b) 1% to 10% one or more dissolved polyvinyl caprolactam-polyvinyl acetate-polyethylene glycol graft copolymer, by weight based on the weight of the solution;
wherein said aqueous solution has complex viscosity of 20 mPa*s or less at 25° C.

2. The aqueous composition of claim 1, wherein the polyvinyl caprolactam-polyvinyl acetate-polyethylene glycol graft copolymer has a polyethylene glycol backbone with one or two side chains; wherein the polyethylene backbone has average molecular weight of 3,000 to 10,000; wherein each of the one or two side chains is a random copolymer of vinyl acetate and N-vinyl caprolactam.

3. The aqueous composition of claim 2, wherein the polyvinyl caprolactam-polyvinyl acetate-polyethylene glycol graft copolymer comprises, by weight based on the weight of the polyvinyl caprolactam-polyvinyl acetate-polyethylene glycol graft copolymer:
(A) the polyethylene backbone in an amount of 7% to 25%;
(B) polymerized units of the vinyl acetate in an amount of 15% to 50%; and
(C) polymerized units of the N-vinyl caprolactam in an amount of 30% to 80%.

4. The aqueous composition of claim 3, wherein the polyvinyl caprolactam-polyvinyl acetate-polyethylene glycol graft copolymer has average molecular weight of 70,000 to 200,000.

5. The aqueous composition of claim 1, wherein the cellulose derivative is selected from the group consisting of methylcellulose, hydroxypropyl methylcellulose, and mixtures thereof.

6. The aqueous composition of claim 1, wherein the aqueous composition has complex viscosity at 37° C. of 30 mPa*s or higher.
